(19) United States
(12) Patent Application Publication
(10) Pub. No.: US 2025/0388525 A1
(43) Pub. Date: Dec. 25, 2025

(54) TEMPERATURE CONTROL FOR HYDRODEOXYGENATION REACTIONS

(71) Applicants: Johnson Matthey Public Limited Company, London (GB); Virent, Inc., Madison, WI (US)

(72) Inventors: Ian Campbell, London (GB); Rauf Edward John Gearing, London (GB); Matthew Van Straten, Madison, WI (US); Brian Blank, Madison, WI (US); Brice Dally, Madison, WI (US); George Tyson, Madison, WI (US); Andrew Held, Madison, WI (US)

(21) Appl. No.: 19/244,884

(22) Filed: Jun. 20, 2025

Related U.S. Application Data
(60) Provisional application No. 63/662,839, filed on Jun. 21, 2024.

(51) Int. Cl. C07C 1/24

(52) U.S. Cl. CPC C07C 1/24

(57) ABSTRACT

The present disclosure provides systems and methods for hydrodeoxygenation (HDO) and other related reactions. A feed stream comprising an oxygenated hydrocarbon can be reacted in a first HDO reactor to produce an intermediate stream. The intermediate stream can be reacted in a second HDO reactor to produce an HDO product stream. The HDO product stream can be fractionated to produce a first HDO vapor product stream and a first HDO liquid product stream. The feed stream can be heated with the first HDO vapor product stream via a first heat exchanger upstream of the first HDO reactor to an inlet temperature for the HDO reactor at which the oxygenated hydrocarbon is thermally stable. At least part of the first HDO liquid product stream can be recycled to mix with the feed stream upstream of the first heat exchanger.

### CROSS-REFERENCE TO RELATED APPLICATIONS

This application claims priority to U.S. Provisional Patent Application No. 63/662,839, filed Jun. 21, 2024, the entirety of which is incorporated herein by reference.

### BACKGROUND

Bioreforming processes can produce aromatic hydrocarbons and other useful compounds from biomass feedstocks, including cellulose, hemicellulose, and lignin. For instance, cellulose and hemicellulose can be used as feedstock for various bioreforming processes, including aqueous phase reforming (APR) and hydrodeoxygenation (HDO)—catalytic reforming processes that, when integrated with hydrogenation, can convert cellulose and hemicellulose into an array of products, including hydrogen, liquid fuels, aromatics, kerosene, diesel fuel, lubricants, and fuel oils, among others. In addition, catalytic acid condensation (AC) can be used to convert oxygenates (e.g., generated by HDO) or other compounds into hydrocarbons.

APR and HDO methods and techniques are described in U.S. Pat. Nos. 6,699,457; 6,964,757; 6,964,758; and 7,618,612 (all to Cortright et al., entitled “Low-Temperature Hydrogen Production from Oxygenated Hydrocarbons”); U.S. Pat. No. 6,953,873 (to Cortright et al., entitled “Low-Temperature Hydrocarbon Production from Oxygenated Hydrocarbons”); and U.S. Pat. Nos. 7,767,867; 7,989,664; and 8,198,486 (all to Cortright, entitled “Methods and Systems for Generating Polyols”), all of which are incorporated herein by reference. Various other APR and HDO methods and techniques are also described in U.S. Pat. Nos. 8,053,615; 8,017,818;7,977,517; 8,362,307; 8,367,882; and 8,455,705 (all to Cortright and Blommel, entitled “Synthesis of Liquid Fuels and Chemicals from Oxygenated Hydrocarbons”); U.S. Pat. No. 8,231,857 (to Cortright, and entitled “Catalysts and Methods for Reforming Oxygenated Compounds”); U.S. Pat. No. 8,350,108 (to Cortright et al., entitled “Synthesis of Liquid Fuels from Biomass”); and International Patent Application No. PCT/US2008/056330 (to Cortright and Blommel, entitled “Synthesis of Liquid Fuels and Chemicals from Oxygenated Hydrocarbons” and published as WO2008109877A1), all of which are incorporated herein by reference.

### SUMMARY OF THE INVENTION

Some aspects of the present disclosure provide a method for producing an oxygenate product. The method can include (i) reacting a feed stream that includes an oxygenated hydrocarbon in a first hydrodeoxygenation (HDO) reactor with hydrogen in the presence of a first HDO catalyst to produce an intermediate stream, the feed stream having an inlet temperature at which the oxygenated hydrocarbon is thermally stable. The method can further include (ii) reacting the intermediate stream in a second hydrodeoxygenation (HDO) reactor with hydrogen in the presence of a second HDO catalyst to produce an HDO product stream; and (iii) fractionating the HDO product stream to produce a first HDO vapor product stream and a first HDO liquid product stream. The method can further include (iv) heating the feed stream with the first HDO vapor product stream via a first heat exchanger upstream of the first HDO reactor; and (v) recycling at least part of the first HDO liquid product stream to mix with the feed stream upstream of the first heat exchanger, to continue (i).

The feed stream can further comprise hydrogen.

The feed stream including the hydrogen can be preheated at a first preheater upstream of the first heat exchanger and upstream of mixing with the recycled at least part of the first HDO liquid product stream.

The first preheater can heat the feed stream to a temperature of less than about 240° C.

The feed stream can be further heated by a second preheater downstream of the first heat exchanger and upstream of the first HDO reactor.

The inlet temperature of the feed stream at the first HDO reactor can be about 200° C. to about 280° C.

The inlet temperature of the feed stream at the first HDO reactor can be less than about 277° C.

The first HDO liquid product stream can be fractionated to produce a second HDO vapor product stream and a second HDO liquid product stream, wherein one or more of the second HDO vapor product stream or the second HDO liquid product stream include the oxygenate product.

Recycling the at least part of the first HDO liquid product stream at (v) can include recycling at least part of the second HDO liquid product stream.

Prior to (iv), the first HDO vapor product stream can be, at a second heat exchanger, with a liquid product stream from one or more of the first or second HDO reactors.

The second heat exchanger can be selectively bypassed with a first part of the first HDO vapor product stream and the first part of the first HDO vapor product stream can be remixed with a second part of the first HDO vapor product stream that is cooled at the second heat exchanger.

Some aspects of the present disclosure provide a method for producing a C₄₊ compound. A HDO vapor product stream and a HDO liquid product stream can be produced. At least a portion of one or more of the HDO vapor product stream or the HDO liquid product stream can be reacted in the presence of a condensation catalyst to produce the C4+ compound.

Some aspects of the present disclosure provide a method for producing a C4+ compound. A HDO vapor product stream and a HDO liquid product stream can be produced by: (i) reacting a feed stream that includes an oxygenated hydrocarbon in a hydrodeoxygenation (HDO) reactor train with hydrogen in the presence of one or more HDO catalysts to produce an HDO product stream; (ii) fractionating the HDO product stream to produce the HDO vapor product stream and the HDO liquid product stream; and one or more of: (iii) heating the feed stream with the first HDO vapor product stream via a first heat exchanger upstream of the HDO reactor train; or (iv) recycling at least part of the first HDO liquid product stream to mix with the feed stream, to continue (i). At least a portion of one or more of the HDO vapor product stream or the HDO liquid product stream can be reacted in the presence of a condensation catalyst to produce the C4+ compound.

Producing the HDO product stream can include: reacting the feed stream in a first HDO reactor with hydrogen in the presence of a first HDO catalyst to produce an intermediate stream; and reacting the intermediate stream in a second HDO reactor with hydrogen in the presence of a second HDO catalyst to produce the HDO product stream.

The intermediate stream can be heated with a second heat exchanger upstream of the second HDO reactor.

The feed stream can be heated with the first HDO vapor product stream via a first heat exchanger upstream of the HDO reactor train and recycling at least part of the first HDO liquid product stream to mix with the feed stream, to continue (i).

The HDO liquid product stream can be flashed to produce a second HDO vapor product stream and a second HDO liquid product stream. The at least a portion of one or more of the HDO vapor product stream or the HDO liquid product stream can include one or more of the second HDO vapor product stream or the second HDO liquid product stream.

Recycling at least part of the first HDO liquid product stream to mix with the feed stream can include recycling the second HDO liquid product stream to mix with the feed stream.

Some aspects of the present disclosure provide a system for producing a C₄₊ compound. A first hydrodeoxygenation (HDO) reactor can include a first HDO catalyst and can be configured to receive a first HDO feed stream and to provide a first HDO effluent stream. A second HDO reactor can include a second HDO catalyst and can be configured to receive the first HDO effluent stream from the first HDO reactor as a second HDO inlet stream and to provide a second HDO effluent stream. A recycle path can be configured to direct a liquid stream separated from the second HDO effluent stream into an initial feed stream to provide the first HDO feed stream. A first heat exchanger can be configured to heat the first HDO feed stream upstream of the first HDO reactor using a vapor stream separated from the second HDO effluent stream.

An HDO product separator can be configured to receive the second HDO effluent stream to separate the vapor stream from the second HDO effluent stream and provide an intermediate liquid stream from the second HDO effluent stream. An HDO product flash drum can be configured to receive the intermediate liquid stream to separate the liquid stream for the recycle path from a vapor product stream.

A hydrogenation (HYD) reactor train can be configured to provide a hydrogenation product as at least part of the first HDO feed stream. An acid condensation (AC) reactor train can be configured to receive the vapor product stream from the HDO product flash drum for condensation reactions to produce the C4+ compound.

### BRIEF DESCRIPTION OF DRAWINGS

FIG. 1 shows an example system for converting biomass to liquid hydrocarbons (e.g. fuel compounds) or other products.

FIG. 2 shows an example HDO reactor train according to an example of the disclosed technology.

### DETAILED DESCRIPTION

The present disclosure relates to processes and systems for HDO reactions, including as can be implemented downstream of hydrogenation reactions and upstream of AC reactions. In one aspect, the present disclosure provides improved temperature control for inlet streams for HDO reactors, including as can improve overall yield of conversion of sugar feeds to liquid hydrocarbon (e.g., C₄₊) products and reduce coke deposits on AC catalysts.

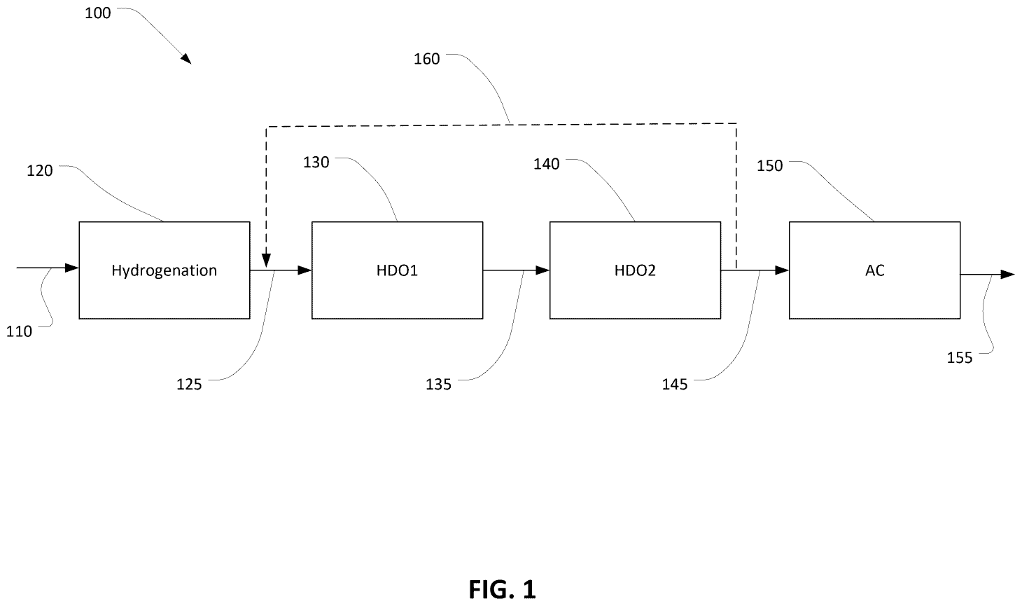

Generally, the technology disclosed herein can be used to improve HDO processing for a wide range of feed streams. As an example context, FIG. 1 illustrates a catalytic reactor system 100 for processing biomass feed streams into liquid hydrocarbons (or other products). The particular system of FIG. 1 should not be viewed as limiting however, as a wide variety of systems can be implemented to provide a feed stream to an HDO reactor system (e.g., as variously disclosed in U.S. Pat. Nos. 6,699,457; 6,964,757; 6,964,758; 7,618,612; 6,953,873; 7,767,867; 7,989,664; 6,953,873; 7,767,867; 7,989,664; 8,198,486; 8,053,615; 8,017,818; 7,977,517; 8,362,307; 8,367,882; 8,455,705 8,231,857; and 8,350,108; in International Patent Publication WO2008109877A1; or as otherwise known in the art).

The term “biomass” refers to, without limitation, organic materials produced by plants (such as leaves, roots, seeds and stalks), and microbial and animal metabolic wastes. Common biomass sources include: (1) agricultural residues, including corn stover, straw, seed hulls, sugarcane leavings, bagasse, nutshells, cotton gin trash, and manure from cattle, poultry, and hogs; (2) wood materials, including wood or bark, sawdust, timber slash, and mill scrap; (3) municipal solid waste, including recycled paper, waste paper and yard clippings; (4) algae-derived biomass, including carbohydrates and lipids from microalgae (e.g., Botryococcus braunii, Chlorella, Dunaliella tertiolecta, Gracilaria, Pleurochyrsis camerae, and Sargassum) and macroalgae (e.g., seaweed); (5) energy crops, including poplars, willows, switch grass, miscanthus, sorghum, alfalfa, prairie bluestream, corn, soybean, and the like; and (6) related partially pre-processed products (e.g., corn syrup of various purities). The term also refers to the primary building blocks of the above, namely, lignin, cellulose, hemicellulose and carbohydrates, such as saccharides, sugars (e.g., glucose, sucrose, etc.) and starches, among others.

In the example reactor system 100 of FIG. 1, a feed stream 110 including biomass is introduced to a hydrogenation (HYD) reactor 120 for catalytic hydrogenation. A product stream 125 from the HYD reactor 120 is introduced to an HDO reactor train to produce an HDO product stream. In some examples, the HDO reactor train can include a first hydrodeoxygenation (HDO1) reactor 130 for catalytic HDO reactions to produce a first intermediate stream 135. The first intermediate stream 135 can then be introduced to a second hydrodeoxygenation (HDO2) reactor 140 of the HDO reactor train for further catalytic HDO reactions to produce a second intermediate stream 145. As appropriate for production of desired compounds, the second intermediate stream 145 can then be provided to an acid condensation (AC) reactor 150 for catalytic AC reactions (e.g., after separation, vaporization, or other treatment) to produce a product stream 155.

For clarity of presentation, the catalytic reactor system 100 is presented with single blocks to represent various reactors, and with single respective feed, intermediate, and product streams for those reactors. It should be recognized that a variety of initial, intermediate, and post-processing operations can be implemented (e.g., heating, cooling, separation, recycle, etc.), that any or all of the reactors illustrated can be implemented as a set of one or more reactors in a reactor train (e.g., for in-series, successive catalytic reactions), and that other variations are also possible as recognized by those of skill in the art or discussed in the various publications incorporated herein by reference.

### HYD Catalyst and HYD Reactions

Various processes are known for hydrogenating sugars, furfurals, carboxylic acids, ketones, and furans to their corresponding alcohol form, including those disclosed by: B.S. Kwak et al. (WO2006/093364A1 and WO 2005/021475A1), involving the preparation of sugar alditols from monosaccharides by hydrogenation over a ruthenium catalyst; and Elliot et al. (U.S. Pat. Nos. 6,253,797 and 6,570,043), disclosing the use of a nickel and rhenium free ruthenium catalyst on a more than 75% rutile titania support to convert sugars to sugar alcohols, all incorporated herein by reference. Other suitable ruthenium catalysts are described by Arndt et al. in published U.S. patent application 2006/0009661 (filed Dec. 3, 2003), and Arena in U.S. Pat. No. 4,380,679 (filed Apr. 12, 1982), U.S. Pat. No. 4,380,680 (filed May 21, 1982), U.S. Pat. No. 4,503,274 (filed Aug. 8, 1983), U.S. Pat. No. 4,382,150 (filed Jan. 19, 1982), and U.S. Pat. No. 4,487,980 (filed Apr. 29, 1983), all incorporated herein by reference. The hydrogenation catalyst generally includes Cu, Re, Ni, Fe, Co, Ru, Pd, Rh, Pt, Os, Ir, and alloys or combinations thereof, either alone or with promoters such as W, Mo, Au, Ag, Cr, Zn, Mn, Sn, B, P, Bi, and alloys or combinations thereof. The hydrogenation catalyst may also include any one of the supports further described below, and depending on the desired functionality of the catalyst. Other effective hydrogenation catalyst materials include either supported nickel or ruthenium modified with rhenium. In general, the hydrogenation reaction is carried out at hydrogenation temperatures of between about 80° C. to 250° C., and hydrogenation pressures in the range of about 100 psig to 2000 psig. The hydrogen used in the reaction may include in situ generated APR hydrogen, external hydrogen, recycled hydrogen, or a combination thereof.

The hydrogenation catalyst may also include a supported Group VIII metal catalyst and a metal sponge material, such as a sponge nickel catalyst. Activated sponge nickel catalysts (e.g., Raney nickel) are a well-known class of materials effective for various hydrogenation reactions. One type of sponge nickel catalyst is the type A7063 catalyst available from Activated Metals and Chemicals, Inc., Sevierville, Tenn. The type A7063 catalyst is a molybdenum promoted catalyst, typically containing approximately 1.5% molybdenum and 85% nickel. The use of the sponge nickel catalyst with a feedstock comprising xylose and dextrose is described by M. L. Cunningham et al. in U.S. Pat. No. 6,498,248, filed Sep. 9, 1999, incorporated herein by reference. The use of a Raney nickel catalyst with hydrolyzed corn starch is also described in U.S. Pat. No. 4,694,113, filed Jun. 4, 1986, and incorporated herein by reference.

The preparation of suitable Raney nickel hydrogenation catalysts is described by A. Yoshino et al. in published U.S. patent application 2004/0143024, filed Nov. 7, 2003, incorporated herein by reference. The Raney nickel catalyst may be prepared by treating an alloy of approximately equal amounts by weight of nickel and aluminum with an aqueous alkali solution, e.g., containing about 25 wt. % of sodium hydroxide. The aluminum is selectively dissolved by the aqueous alkali solution leaving particles having a sponge construction and composed predominantly of nickel with a minor amount of aluminum. Promoter metals, such as molybdenum or chromium, may be also included in the initial alloy in an amount such that about 1-2 wt. % remains in the sponge nickel catalyst.

In another embodiment, the hydrogenation catalyst is prepared by impregnating a suitable support material with a solution of ruthenium (III) nitrosylnitrate, ruthenium (III) nitrosylnitrate, or ruthenium (III) chloride in water to form a solid that is then dried for 13 hours at 120° C. in a rotary ball oven (residual water content is less than 1% by weight). The solid is then reduced at atmospheric pressure in a hydrogen stream at 300° C. (uncalcined) or 400° C. (calcined) in the rotary ball furnace for 4 hours. After cooling and rendering inert with nitrogen, the catalyst may then be passivated by passing over 5% by volume of oxygen in nitrogen for a period of 120 minutes.

In yet another embodiment, the hydrogenation reaction is performed using a catalyst comprising a nickel-rhenium catalyst or a tungsten-modified nickel catalyst. One example of a suitable hydrogenation catalyst is the carbon-supported nickel-rhenium catalyst composition disclosed by Werpy et al. in U.S. Pat. No. 7,038,094, filed Sep. 30, 2003, and incorporated herein by reference.

In other embodiments, it may also be desirable to convert the starting oxygenated hydrocarbon, such as a sugar, sugar alcohol or other polyhydric alcohol, to a smaller molecule that can be more readily converted to the desired oxygenates, such as by hydrogenolysis. Such smaller molecules may include primary, secondary, tertiary or polyhydric alcohols having less carbon atoms than the originating oxygenated hydrocarbon. Various processes are known for such hydrogenolysis reactions, including those disclosed by: Werpy et al. in U.S. Pat. No. 6,479,713 (filed Oct. 23, 2001), U.S. Pat. No. 6,677,385 (filed Aug. 6, 2002), U.S. Pat. No. 6,6841,085 (filed Oct. 23, 2001) and U.S. Pat. No. 7,083,094 (filed Sep. 30, 2003), all incorporated herein by reference and describing the hydrogenolysis of 5 and 6 carbon sugars and sugar alcohols to propylene glycol, ethylene glycol and glycerol using a rhenium-containing multi-metallic catalyst. Other systems include those described by Arena in U.S. Pat. No. 4,401,823 (filed May 18, 1981) directed to the use of a carbonaceous pyropolymer catalyst containing transition metals (such as chromium, molybdenum, tungsten, rhenium, manganese, copper, cadmium) or Group VIII metals (such as iron, cobalt, nickel, platinum, palladium, rhodium, ruthenium, iridium and osmium) to produce alcohols, acids, ketones, and ethers from polyhydroxylated compounds, such as sugars and sugar alcohols, and U.S. Pat. No. 4,496,780 (filed Jun. 22, 1983) directed to the use of a catalyst system having a Group VIII noble metal on a solid support with an alkaline earth metal oxide to produce glycerol, ethylene glycol and 1,2-propanediol from carbohydrates, each incorporated herein by reference. Another system includes that described by Dubeck et al. in U.S. Pat. No. 4,476,331 (filed Sep. 6, 1983) directed to the use of a sulfide-modified ruthenium catalyst to produce ethylene glycol and propylene glycol from larger polyhydric alcohols, such as sorbitol, also incorporated herein by reference. Other systems include those described by Saxena et al., “Effect of Catalyst Constituents on (Ni, Mo and Cu)/Kieselguhr-Catalyzed Sucrose Hydrogenolysis,” Ind. Eng. Chem. Res. 44, 1466-1473 (2005), describing the use of Ni, W, and Cu on a kieselguhr support, incorporated herein by reference.

In one embodiment, the hydrogenolysis catalyst includes Cr, Mo, W, Re, Mn, Cu, Cd, Fe, Co, Ni, Pt, Pd, Rh, Ru, Ir, or Os, and alloys or combinations thereof, either alone or with promoters such as Au, Ag, Cr, Zn, Mn, Sn, Bi, B, O and alloys or combinations thereof. Other effective hydrogenolysis catalyst materials may include the above metals combined with an alkaline earth metal oxide or adhered to catalytically active support, such as kieselguhr, or any one of the supports further described below.

The process conditions for carrying out the hydrogenolysis reaction will vary depending on the type of feedstock and desired products. In general, the hydrogenolysis reaction is conducted at temperatures of at least 110° C., or between 110° C. and 300° C., or between 170° C. and 240° C. The reaction should also be conducted under basic conditions, preferably at a pH of about 8 to about 13, or at a pH of about 10 to about 12. The reaction should also be conducted at pressures of between about 10 psig and 2400 psig, or between about 250 psig and 2000 psig, or between about 700 psig and 1600 psig. The hydrogen used in the reaction may include APR hydrogen, external hydrogen, recycled hydrogen, or a combination thereof.

### HDO Catalyst and HDO Reaction

The term “hydrodeoxygenation catalyst” (HDO catalyst) refers to a catalyst that catalyzes a process that removes oxygen from oxygen-containing compounds in the presence of hydrogen. Suitable HDO catalysts and processes include, for example, those described in WO 2014/152370 and WO/2023/064565, all of which are incorporated herein by reference.

In some embodiments, the HDO catalyst is composed of a heterogeneous catalyst having one or more materials capable of catalyzing a reaction between hydrogen and a feedstock solution to remove one or more of the oxygen atoms from the feedstock solution to produce one or more oxygenate. In some embodiments, the HDO catalyst is composed of one or more metal adhered to a support and may include, without limitation, Cu, Re, Fe, Ru, Ir, Co, Rh, Pt, Pd, Ni, W, Os, Mo, Ag, Au, alloys and combinations thereof. The HDO catalyst may include these elements alone or in combination with one or more promoters, such as Mn, Cr, Mo, W, V, Nb, Ta, Ti, Zr, Y, La, Sc, Zn, Cd, Ag, Au, Sn, Ge, P, Al, Ga, In, Tl, and combinations thereof. In some embodiments, the HDO catalyst includes Pt, Ru, Cu, Re, Co, Fe, Ni, W or Mo. In some embodiments, the HDO catalyst includes Fe or Re and at least one transition metal selected from Ir, Ni, Pd, P, Rh, and Ru. In some embodiments, the HDO catalyst includes Fe, Re and at least Cu or one Group VIIIB transition metal. In some embodiments, the metal of the HDO catalyst comprises Pd, W, Mo, Ni, Pt, Ru, or a combination thereof. In some embodiments, the HDO catalyst comprises a promoter. As an example, the promoter of the deoxygenation catalyst can comprise Sn, W, or a combination thereof. The support may include a nitride, carbon, silica, alumina, zirconia, titania, vanadia, ceria, zinc oxide, chromia, boron nitride, heteropolyacids, kieselguhr, hydroxyapatite, or a mixture thereof. In some embodiments, the support comprises zirconia.

The aqueous feed stream is reacted with hydrogen in the presence of the HDO catalyst at temperatures, pressures, and weight hourly space velocities effective to produce the desired oxygenate products. The specific oxygenates produced will depend on various factors, including the feedstock solution, reaction temperature, reaction pressure, water concentration, hydrogen concentration, the reactivity of the catalyst, and the flow rate of the feedstock solution as it affects the space velocity (the mass/volume of reactant per unit of catalyst per unit of time), gas hourly space velocity (GHSV), and weight hourly space velocity (WHSV). For example, an increase in flow rate, and thereby a reduction of the feed stream exposure to the HDO catalyst over time, will limit the extent of the reactions that may occur, thereby causing increased yield for higher level di-and tri-oxygenates, with a reduction in ketone, alcohol, and cyclic ether yields.

In general, the reaction may include a temperature gradient to allow partial deoxygenation of the oxygenated hydrocarbon at temperatures below the caramelization point of a feedstock, from which the aqueous feed stream is generated. Including a temperature gradient helps prevent the oxygenated hydrocarbons in the feed stream from condensing (e.g., caramelizing) on the catalyst and creating a substantial pressure drop across the reactor, which can lead to inoperability of the reactor. The caramelization point, and therefore the required temperature gradient, will vary depending on the feedstock. In one embodiment, the temperature gradient is from about 170° C. to 300° C. or between about 200° C. to 290° C. In another embodiment, a temperature gradient is not employed.

Operating pressures up to about 2000 psig can be used to help maintain the carbon backbone, minimize the amount of light organic acids and ketones that are formed, and increase the product selectivity towards alcohols. At increased operating pressures, the thermodynamics of the reaction can favor alcohols to ketones and organic acids, thereby shifting the product selectivity, maintaining the carbon backbone, and improving product yields. In this regard, light organic acids may be particularly undesirable products as they are highly corrosive. Producing fewer light organic acids can provide more flexibility with regards to materials of construction of a reactor system because corrosion is less of an issue.

The reaction temperature and pressures are preferably selected to maintain at least a portion of a feedstock, from which the aqueous feed stream is generated, in the liquid phase at the reactor inlet. It is recognized, however, that temperature and pressure conditions may also be selected to more favorably produce the desired products in the vapor-phase. In general, the reaction should be conducted at process conditions wherein the thermodynamics of the proposed reaction are favorable. For instance, the minimum pressure required to maintain a portion of the feedstock in the liquid phase will likely vary with the reaction temperature. As temperatures increase, higher pressures will generally be required to maintain the feedstock in the liquid phase, if desired. Pressures above that required to maintain the feedstock in the liquid phase (i.e., vapor-phase) are also suitable operating conditions.

In condensed phase liquid reactions, the pressure within the reactor generally must be sufficient to maintain the reactants in the condensed liquid phase at the reactor inlet. For example, for liquid phase reactions, the reaction temperature should be greater than about 100° C., or 120° C., or 150° C., or 180° C., or 200° C., and less than about 300° C., or 290° C., or 270° C., or 250° C., or 220° C. Similarly, the reaction pressure should be greater than about 70 psig, or 145 psig, or 300 psig, or 500 psig, or 750 psig, or 1050 psig, and less than about 2000 psig, or 1950 psig, or 1900 psig, or 1800 psig. In one embodiment, the reaction temperature is between about 120° C. and 300° C., or between about 200° C. and 300° C., or between about 270° C. and 290° C., and the reaction pressure is between about 145 and 1950 psig, or between about 1000 and 1900 psig, or between about 1050 and 1800 psig.

For vapor phase reactions, the reaction should be carried out at a temperature where the vapor pressure of the oxygenated hydrocarbon is at least about 0.1 atm, preferably higher (e.g., 350 psi), and the thermodynamics of the reaction are favorable. This temperature will vary depending upon the specific oxygenated hydrocarbon compound used, but is generally greater than about 100° C., or 120° C., or 250° C., and less than about 600° C., or 500° C., or 400° C. for vapor phase reactions. In one embodiment, the reaction temperature is between about 120° C. and about 500° C., or between about 250° C. and about 400° C.

In general, the HDO reaction should be conducted under conditions where the residence time of the aqueous feed stream over the catalyst is appropriate to generate the desired products. For example, the WHSV for the reaction may be at least 0.01 gram of oxygenated hydrocarbon per gram of catalyst per hour (g/g-hr). In some embodiments, the WHSV for the HDO reaction is 0.01 to about 40.0 g/g-hr, such as about 0.05 to about 40.0, about 1.0 to about 40.0, about 5.0 to about 40.0, or about 1.0 to about 20.0 g/g-hr. The WHSV can be, for example, about 0.05, 0.1, 0.25, 0.5, 0.75, 1.0, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6, 7, 8, 9, 10, 11, 12, 13, 14, 15, 20, 25, 30, 35, or 40 g/g-hr.

In some embodiments, the amount of hydrogen fed to the HDO reaction ranges from 0-2400%, 5-2400%, 10-2400%, 15-2400%, 20-2400%, 25-2400%, 30-2400%, 35-2400%, 40-2400%, 45-2400%, 50-2400%, 55-2400%, 60-2400%, 65-2400%, 70-2400%, 75-2400%, 80-2400%, 85-2400%, 90-2400%, 95-2400%, 98-2400%, 100-2400%, 200-2400%, 300-2400%, 400-2400%, 500-2400%, 600-2400%, 700-2400%, 800-2400%, 900-2400%, 1000-2400%, 1100-2400%, or 1150-2400%, or 1200-2400%, or 1300-2400%, or 1400-2400%, or 1500-2400%, or 1600-2400%, or 1700-2400%, or 1800-2400%, or 1900-2400%, or 2000-2400%, or 2100-2400%, or 2200-2400%, or 2300-2400%, based on the total number of moles of the oxygenated hydrocarbon(s) in the feedstock, including all intervals between. The hydrogen may be external hydrogen or recycled hydrogen. The term “external H₂” refers to hydrogen that does not originate from the feedstock solution but is added to the reactor system from an external source. The term “recycled H₂” refers to unconsumed hydrogen, which is collected and then recycled back into the reactor system for further use.

### AC Catalyst and AC Reactions

In some examples, reacting the HDO product stream (or another product stream) in the presence of a condensation catalyst (i.e., in AC reactor(s)) can produce a C₄₊ compound. The C₄₊ compound can include a member selected from the group consisting of C₄₊ alcohol, C₄₊ ketone, C₄₊ alkane, C₄₊ alkene, C₅₊ cycloalkane, C₅₊ cycloalkene, aryl, fused aryl, and a mixture thereof. In one exemplary embodiment, the C₄₊ alkane comprises a branched or straight chain C₄₋₃₀ alkane, or a branched or straight chain C₄₋₉, C₇₋₁₄, C₁₂₋₂₄ alkane, or a mixture thereof. In another exemplary embodiment, the C₄₊ alkene comprises a branched or straight chain C₄₋₃₀ alkene, or a branched or straight chain C₄₋₉, C₇₋₁₄, C₁₂₋₂₄ alkene, or a mixture thereof. In another exemplary embodiment, the C₅₊ cycloalkane comprises a mono-substituted or multi-substituted C₅₊ cycloalkane, and at least one substituted group is a branched C₃₊ alkyl, a straight chain C₁₊ alkyl, a branched C₃₊ alkylene, a straight chain C₁₊ alkylene, a phenyl, or a combination thereof, or a branched C₃₋₁₂ alkyl, a straight chain C₁₋₁₂ alkyl, a branched C₃₋₁₂ alkylene, a straight chain C₁₋₁₂ alkylene, a phenyl, or a combination thereof, or a branched C₃₋₄ alkyl, a straight chain C₁₋₄ alkyl, a branched C₃₋₄ alkylene, straight chain C₁₋₄ alkylene, a phenyl, or a combination thereof. In another exemplary embodiment, the C₅₊ cycloalkene comprises a mono-substituted or multi-substituted C₅₊ cycloalkene, and at least one substituted group is a branched C₃₊ alkyl, a straight chain C₁₊alkyl, a branched C₃₊ alkylene, a straight chain C₂₊ alkylene, a phenyl, or a combination thereof, or a branched C₃₋₁₂ alkyl, a straight chain C₁₋₁₂ alkyl, a branched C₃₋₁₂ alkylene, a straight chain C₂₋₁₂ alkylene, a phenyl, or a combination thereof, or a branched C₃₋₄ alkyl, a straight chain C₁₋₄ alkyl, a branched C₃₋₄ alkylene, straight chain C₂₋₄ alkylene, a phenyl, or a combination thereof. In another exemplary embodiment, the aryl comprises an unsubstituted aryl, or a mono-substituted or multi-substituted aryl, and at least one substituted group is a branched C₃₊ alkyl, a straight chain C₁₊ alkyl, a branched C₃₊ alkylene, a straight chain C₂₊ alkylene, a phenyl, or a combination thereof, or a branched C₃₋₁₂ alkyl, a straight chain C₁₋₁₂ alkyl, a branched C₃₋₁₂ alkylene, a straight chain C₂₋₁₂ alkylene, a phenyl, or a combination thereof, or a branched C₃₋₄ alkyl, a straight chain C₁₋₄ alkyl, a branched C₃₋₄ alkylene, a straight chain C₂₋₄ alkylene, a phenyl, or a combination thereof. In another exemplary embodiment, the fused aryl comprises an unsubstituted fused aryl, or a mono-substituted or multi-substituted fused aryl, and at least one substituted group is a branched C₃₊ alkyl, a straight chain C₁₊ alkyl, a branched C₃₊ alkylene, a straight chain C₂₊ alkylene, a phenyl, or a combination thereof, or a branched C₃₋₄ alkyl, a straight chain C₁₋₄ alkyl, a branched C₃₋₄ alkylene, a straight chain C₂₋₄ alkylene, a phenyl, or a combination thereof. In another exemplary embodiment, the C₄₊ alcohol comprises a compound according to the formula R¹-OH, wherein R¹ is a branched C₄₊ alkyl, straight chain C₄₊ alkyl, a branched C₄₊ alkylene, a straight chain C₄₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl, or a combination thereof.

In another exemplary embodiment of method of making the C₄₊ compound, the C₄₊ ketone comprises a compound according to the formula

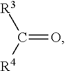

wherein R³ and R⁴ are independently a branched C₃₊ alkyl, a straight chain C₁₊ alkyl, a branched C₃₊ alkylene, a straight chain C₂₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl, or a combination thereof. Examples of desirable C₄₊ ketones include, without limitation, butanone, pentanone, hexanone, heptanone, octanone, nonanone, decanone, undecanone, dodecanone, tridecanone, tetradecanone, pentadecanone, hexadecanone, heptyldecanone, octyldecanone, nonyldecanone, eicosanone, uncicosanone, doeicosanone, trieicosanone, tetracicosanone, or isomers thereof.

The condensation catalyst is generally a catalyst capable of forming longer chain compounds by linking two molecules (e.g., oxygen containing species or other functionalized compounds, including olefins) through a new carbon-carbon bond, and converting the resulting compound to a hydrocarbon, alcohol, or ketone. In some embodiments, the condensation catalyst is an acid condensation catalyst. The condensation catalyst may include, without limitation, carbides, nitrides, zirconia, alumina, silica, aluminosilicates, phosphates, zeolites (e.g., ZSM-5, ZSM-11, ZSM-12, ZSM-22, ZSM-23, ZSM-35 and ZSM-48), titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, acid modified resins, base modified resins, and combinations thereof. The condensation catalyst may include the above alone or in combination with a modifier, such as Ce, La, Y, Sc, P, B, Bi, Li, Na, K, Rb, Cs, Mg, Ca, Sr, Ba, and combinations thereof. The condensation catalyst may also include a metal, such as Cu, Ag, Au, Pt, Ni, Fe, Co, Ru, Zn, Cd, Ga, In, Rh, Pd, Ir, Re, Mn, Cr, Mo, W, Sn, Os, alloys and combinations thereof, to provide a metal functionality.

The condensation catalyst may be self-supporting (i.e., the catalyst does not need another material to serve as a support) or may require a separate support suitable for suspending the catalyst in the reactant stream. One particularly beneficial support is silica, especially silica having a high surface area (greater than 100 square meters per gram), obtained by sol-gel synthesis, precipitation or fuming. In other embodiments, particularly when the condensation catalyst is a powder, the catalyst system may include a binder to assist in forming the catalyst into a desirable catalyst shape. Applicable forming processes include extrusion, pelletization, oil dropping, or other known processes. Zinc oxide, alumina, and a peptizing agent may also be mixed together and extruded to produce a formed material. After drying, this material is calcined at a temperature appropriate for formation of the catalytically active phase, which usually requires temperatures in excess of 450° C.

The condensation catalyst may include one or more zeolite structures comprising cage-like structures of silica-alumina. Zeolites are crystalline microporous materials with well-defined pore structures. Zeolites contain active sites, usually acid sites, which can be generated in the zeolite framework. The strength and concentration of the active sites can be tailored for particular applications. Examples of suitable zeolites for condensing secondary alcohols and alkanes may comprise aluminosilicates, optionally modified with cations, such as Ga, In, Zn, Mo, and mixtures of such cations, as described, for example, in U.S. Pat. No. 3,702,886, which is incorporated herein by reference. As recognized in the art, the structure of the particular zeolite or zeolites may be altered to provide different amounts of various hydrocarbon species in the product mixture. Depending on the structure of the zeolite catalyst, the product mixture may contain various amounts of aromatic and cyclic hydrocarbons.

Examples of suitable zeolite catalysts include ZSM-5, ZSM-11, ZSM-12, ZSM-22, ZSM-23, ZSM-35 and ZSM-48. Zeolite ZSM-5, and the conventional preparation thereof, is described in U.S. Pat. No. 3,702,886; Re. 29,948 (highly siliccous ZSM-5); U.S. Pat. Nos. 4,100,262 and 4,139,600, all incorporated herein by reference. Zeolite ZSM-11, and the conventional preparation thereof, is described in U.S. Pat. No. 3,709,979, which is also incorporated herein by reference. Zeolite ZSM-12, and the conventional preparation thereof, is described in U.S. Pat. No. 3,832,449, incorporated herein by reference. Zeolite ZSM-23, and the conventional preparation thereof, is described in U.S. Pat. No. 4,076,842, incorporated herein by reference. Zeolite ZSM-35, and the conventional preparation thereof, is described in U.S. Pat. No. 4,016,245, incorporated herein by reference. Another preparation of ZSM-35 is described in U.S. Pat. No. 4,107,195, the disclosure of which is incorporated herein by reference. ZSM-48, and the conventional preparation thereof, is taught by U.S. Pat. No. 4,375,573, incorporated herein by reference. Other examples of zeolite catalysts are described in U.S. Pat. No. 5,019,663 and U.S. Pat. No. 7,022,888, also incorporated herein by reference. An exemplary condensation catalyst is a ZSM-5 zeolite modified with Cu, Pd, Ag, Pt, Ru, Re, Ni, Sn, or combinations thereof.

As described in U.S. Pat. No. 7,022,888, which is incorporated herein by reference, the condensation catalyst may be a bifunctional pentasil zeolite catalyst including at least one metallic clement from the group of Cu, Ag, Au, Pt, Ni, Fe, Co, Ru, Zn, Cd, In, Rh, Pd, Ir, Re, Mn, Cr, Mo, W, Sn, Os, alloys and combinations thereof, or a modifier from the group of In, Zn, Fe, Mo, Au, Ag, Y, Sc, Ni, P, Ta, lanthanides, and combinations thereof. The zeolite may have strong acidic sites, and may be used with reactant streams containing an oxygenated hydrocarbon at a temperature of below 580° C. The bifunctional pentasil zeolite may have ZSM-5, ZSM-8 or ZSM- 11 type crystal structure consisting of a large number of 5-membered oxygen-rings (i.e., pentasil rings). In one embodiment the zeolite will have a ZSM-5 type structure.

Alternatively, solid acid catalysts such as alumina modified with phosphates, chloride, silica, and other acidic oxides may be used. Also, sulfated zirconia, phosphated zirconia, titania zirconia, or tungstated zirconia may provide the necessary acidity. Re and Pt/Re catalysts are also useful for promoting condensation of oxygenates to C₅₊ hydrocarbons and/or C₅₊ mono-oxygenates. The Re is sufficiently acidic to promote acid-catalyzed condensation. In certain embodiments, acidity may also be added to activated carbon by the addition of either sulfates or phosphates.

The specific C₄₊ compounds produced will depend on various factors, including, without limitation, the type of oxygenated compounds in the reactant stream, condensation temperature, condensation pressure, the reactivity of the catalyst, and the flow rate of the reactant stream as it affects the space velocity, GHSV, LHSV, and WHSV. In certain embodiments, the reactant stream is contacted with the condensation catalyst at a WHSV that is appropriate to produce the desired hydrocarbon products. In one embodiment the WHSV is at least 0.1 grams of volatile (C₂₊O₁₋₃) oxygenates in the reactant stream per gram catalyst per hour. In another embodiment the WHSV is between 0.1 to 10.0 g/g hr, including a WHSV of 1, 2, 3, 4, 5, 6, 7, 8, 9, 10 g/g hr, and increments between.

In certain embodiments the condensation reaction is carried out at a temperature and pressure at which the thermodynamics of the proposed reaction are favorable. For volatile C₂₊O₁₋₃ oxygenates the reaction may be carried out at a temperature where the vapor pressure of the volatile oxygenates is at least 0.1 atm (and preferably a good deal higher). The condensation temperature will vary depending upon the specific composition of the oxygenated compounds. The condensation temperature will generally be greater than 80° C., or 100° C., or 125° C., or 150° C., or 175° C., or 200° C., or 225° C., or 250° C., and less than 500° C., or 450° C., or 425° C., or 375° C., or 325° C., or 275° C. For example, the condensation temperature may be between 80° C. to 500° C., or between 125° C. to 450° C., or between 250° C. to 425° C. The condensation pressure will generally be greater than 0 psig, or 10 psig, or 100 psig, or 200 psig, and less than 2000 psig, or 1800 psig or, or 1600 psig, or 1500 psig, or 1400 psig, or 1300 psig, or 1200 psig, or 1100 psig, or 1000 psig, or 900 psig, or 700 psig. For example, the condensation pressure may be greater than 0.1 atm, or between 0 and 1500 psig, or between 0 and 1200 psig.

C₄₊ alkanes and C₄₊ alkenes produced from acid condensation can have from 4 to 30 carbon atoms (C₄₊ alkanes and C₄₊ alkenes) and may be branched or straight chained alkanes or alkenes. The C₄₊ alkanes and C₄₊ alkenes may also include fractions of C₄₋₉, C₇₋₁₄, C₁₂₋₂₄ alkanes and alkenes, respectively, with the C₄₋₉ fraction directed to gasoline, the C₇₋₁₆ fraction directed to jet fuels, and the C₁₁₋₂₄ fraction directed to diesel fuel and other industrial applications, such as chemicals. Examples of various C₄₊ alkanes and C₄₊ alkenes include, without limitation, butane, butene, pentane, pentene, 2-methylbutane, hexane, hexene, 2-methylpentane, 3-methylpentane, 2,2-dimethylbutane, 2,3-dimethylbutane, heptane, heptene, octane, octene, 2,2,4,-trimethylpentane, 2,3-dimethyl hexane, 2,3,4-trimethylpentane, 2,3-dimethylpentane, nonane, nonene, decane, decene, undecane, undecene, dodecane, dodecene, tridecane, tridecene, tetradecane, tetradecene, pentadecane, pentadecene, hexadecane, hexadecene, heptyldecane, heptyldecene, octyldecane, octyldecene, nonyldecane, nonyldecene, cicosane, cicosene, uneicosane, uncicosene, docicosane, docicosene, tricicosane, tricicosene, tetracicosane, tetracicosene, and isomers thereof.

C₅₊ cycloalkanes and C₅₊ cycloalkenes produced from acid condensation can have from 5 to 30 carbon atoms and may be unsubstituted, mono-substituted or multi-substituted. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₁₂ alkyl, a straight chain C₁₋₁₂ alkyl, a branched C₃₋₁₂ alkylene, a straight chain C₁₋₁₂ alkylene, a straight chain C₂₋₁₂ alkylene, a phenyl or a combination thereof. By way of further example, at least one of the substituted groups include a branched C₃₋₄ alkyl, a straight chain C₁₋₄ alkyl, a branched C₁₋₄ alkylene, straight chain C₁₋₄ alkylene, straight chain C₂₋₄ alkylene, a phenyl or a combination thereof. Examples of desirable C₅₊ cycloalkanes and C₅₊ cycloalkenes include, without limitation, cyclopentane, cyclopentene, cyclohexane, cyclohexene, methyl-cyclopentane, methyl-cyclopentene, ethyl-cyclopentane, ethyl-cyclopentene, ethyl-cyclohexane, ethyl-cyclohexene, propyl-cyclohexane, butyl-cyclopentane, butyl-cyclohexane, pentyl-cyclopentane, pentyl-cyclohexane, hexyl-cyclopentane, hexyl-cyclohexane, and isomers thereof.

Aryls will generally consist of an aromatic hydrocarbon in either an unsubstituted (phenyl), mono-substituted or multi-substituted form. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₊ alkyl, a straight chain C₁₋₁₂ alkyl, a branched C₃₋₁₂ alkylene, a straight chain C₂₋₁₂ alkylene, a phenyl or a combination thereof. By way of further example, at least one of the substituted groups include a branched C₃₋₄ alkyl, a straight chain C₁₋₄ alkyl, a branched C₃₋₄ alkylene, straight chain C₂₋₄ alkylene, a phenyl or a combination thereof. Examples of various aryls include, without limitation, benzene, toluene, xylene (dimethylbenzene), ethyl benzene, para xylene, meta xylene, ortho xylene, C₉₊ aromatics, butyl benzene, pentyl benzene, hexyl benzene, heptyl benzene, octyl benzene, nonyl benzene, decyl benzene, undecyl benzene, and isomers thereof.

Fused aryls will generally consist of bicyclic and polycyclic aromatic hydrocarbons, in either an unsubstituted, mono-substituted, or multi-substituted form. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₄ alkyl, a straight chain C1₋₄ alkyl, a branched C₃₋₄ alkylene, straight chain C₂₋₄ alkylene, a phenyl or a combination thereof. Examples of various fused aryls include, without limitation, naphthalene, anthracene, and isomers thereof.

Polycyclic compounds will generally consist of bicyclic and polycyclic hydrocarbons, in either an unsubstituted, mono-substituted, or multi-substituted form. Although polycyclic compounds generally include fused aryls, as used herein the polycyclic compounds generally have at least one saturated or partially saturated ring. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₄ alkyl, a straight chain C₁₋₄ alkyl, a branched C₃₋₄ alkylene, straight chain C₂₋₄ alkylene, a phenyl or a combination thereof. Examples of various fused aryls include, without limitation, tetrahydronaphthalene and decahydronaphthalene, and isomers thereof.

The C₄₊ alcohols may also be cyclic, branched or straight chained, and have from 4 to 30 carbon atoms. In general, the C++alcohols may be a compound according to the formula R¹—OH, wherein R¹ is a member selected from a branched C₄₊ alkyl, straight chain C₄₊ alkyl, a branched C₄₊ alkylene, a straight chain C₄₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl or combinations thereof. Examples of desirable C₄₊ alcohols include, without limitation, butanol, pentanol, hexanol, heptanol, octanol, nonanol, decanol, undecanol, dodecanol, tridecanol, tetradecanol, pentadecanol, hexadecanol, heptyldecanol, octyldecanol, nonyldecanol, eicosanol, uneicosanol, docicosanol, trieicosanol, tetracicosanol, or isomers thereof.

In some embodiments, a condensation product stream comprising C₄₊ compounds can be fractionated into various product streams, such as gasoline, jet fuel (kerosene), diesel fuel, and aromatics. For example, the condensation product stream may be passed through a three-phase separator to separate the condensation product stream into an acid condensation gas stream, an organic stream, and an aqueous stream. The organic stream and aqueous stream can be separated by density difference, while the acid condensation gas stream comprising uncondensed gases can be recycled to the acid condensation reactor to generate additional C₄₊ compounds. In some embodiments, a gas transport device, such as a blower or compressor, can be configured in the acid condensation gas stream to control the recycle pressure. In some embodiments, an optional purge stream may also be used to control the pressure of the recycle loop in the acid condensation gas stream. In some embodiments, the aqueous stream is discarded from the process, or further processed in downstream process units.

In some embodiments, the organics stream is fractionated in a distillation column to separate the organic stream into a light product stream and a heavy product stream. In some embodiments, the distillation unit is configured to remove co-boiling contaminants for benzene, toluene, or a combination thereof.

In some embodiments, the distillation column is configured to generate a heavy stream that is free or substantially free of co-boiling non-aromatic contaminants for benzene. The distillation column may remove co-boiling nonaromatic contaminants for benzene by fractionating the organic stream into a C₆₋ stream comprising benzene, co-boiling non-aromatic contaminants for benzene, and lighter products through the light product stream. The distillation column may further fractionate the organic stream into a heavy product stream comprising C₇₊ compounds.

In some embodiments, the distillation column is configured to generate a heavy stream that is free or substantially free of co-boiling nonaromatic contaminants for toluene. The distillation column may remove co-boiling nonaromatic contaminants for toluene by fractionating the organic stream into a C₇₋ or C₈₋ stream comprising toluene, co-boiling nonaromatic contaminants for toluene, and lighter products through the light product stream. The distillation column may further fractionate the organic stream into a heavy product stream comprising C₈₊ or C₉₊ compounds.

In some embodiments, the heavy product stream is fractionated in a distillation column to separate the heavy product stream comprising C₇₊ compounds, C₈₊ compounds, or C₉₊ compounds into the mixed aromatic feed stream and a heavy product stream. In some embodiments, the distillation column is configured to fractionate the heavy product stream into a mixed aromatic feed stream comprising C₇₊ compounds and a heavy product feed stream comprising C₁₁₊ compounds. In some embodiments, the mixed aromatic feed stream comprises C₇₊ compounds, or C₈₊ compounds, or C₉₊ compounds, or C₇₋₁₀ compounds, or C₈₋₁₀ compounds, or C₉₋₁₀ compounds.

In some embodiments, the heavy stream may be further separated for use as kerosene (e.g., C₁₁₋₁₄ as jet fuel use), diesel fuel use (e.g., C₁₂₋₂₄), and lubricants or fuel oils (e.g., C₂₅₊). Alternatively, the heavy stream may be cracked to produce addition fractions for use in gasoline, kerosene, aromatics, and/or diesel fractions.

### Control of HDO Reaction Train

HDO reaction systems are generally of high importance for conversion of sugars to aromatics, with significant impact on overall yield in the conversion of sugar feed to liquid products and in the proportion of the feed deposited downstream (e.g., on the downstream AC catalyst) as coke. The coke rate, for example, can have a direct impact on the size and cost of the equipment needed to regenerate the AC catalyst.

In particular, thermal stability of a feed stream for an HDO reactor can limit the range of inlet temperatures that may be usefully employed for processing of biomass (e.g., in systems as generally represented in FIG. 1). With regard to certain feeds, including corn syrup, hydrogenating an initial feed stream—e.g., to convert sugars to sorbitol—can provide notable improvement in thermal stability. Another feature that can improves the thermal stability of a feed stream for a reactor is the use of a liquid recycle that includes partially converted products from the HDO reactor and that includes little or no sorbitol (e.g., less than 1 wt %).

Accordingly, aspects of the present disclosure can provide improved yields from AC systems and reduced coke rates for AC catalysts through selective control of the temperature of feed streams for HDO reactors and through selective recycle of liquid products from HDO reactions to the feed streams. In particular, some implementations can utilize a separated vapor product from an HDO effluent stream to preheat an HDO feed stream before the feed stream is introduced to the relevant HDO reactor. Some implementations can additionally (or alternatively) recycle a liquid fraction of the HDO effluent stream to be mixed into an HDO feed stream (e.g., as received from upstream HYD reactions) before the feed stream is introduced to the relevant HDO reactor.

In some examples, a preheated HYD product can be mixed with an HDO reactor liquid recycle stream to provide a mixed feed stream for HDO. This initial mixing with the liquid recycle stream can allow heating of the feed stream to an increased temperature before thermal degradation occurs, as compared to unmixed HYD product.

In some implementations, the mixed (or other) feed stream can be heated via heat exchange with an HDO reactor vapor product stream. Before this heat exchange, the HDO reactor vapor product stream may in some cases be cooled to prevent overheating of the feed stream and corresponding thermal degradation. For example, the vapor product stream can be cooled by a condensed HDO reactor product stream, with selective bypass of the vapor product stream around the relevant heat exchanger to provide a target maximum temperature (e.g., 527° F. (275° C.)) after mixing with the cooled vapor product stream. The HDO reactor vapor product stream can be provided at a sufficiently cool temperature to avoid thermal degradation of the mixed HDO reactor feed, while heating the mixed HDO reactor feed to a target intermediate maximum temperature (e.g., 464° F. (240° C.)). Further trim heating of the mixed preheated HDO reactor feed can then be achieved, as appropriate, using thermal oil or other process fluid (e.g., at a temperature of about 550° F. (288° C.)), which can heat the HDO reactor feed to a target final maximum temperature (e.g., 500° F. (260° C.)). Trim heating can be used, for example, to fine tune the inlet temperature to the first HDO reactor, so that it can be gradually increased from start of run (SOR) to end of run (EOR) during operation.

Generally, HDO reactions (and HDO catalyst(s)) can be split between a first reactor and a second reactor. For example, the first reactor may include less catalyst than the second reactor, corresponding to the need to operate the first reactor at lower temperature due to the lower thermal stability of its feed stream. The reactions which occur over the first HDO reactor can generally break down sorbitol from the HYD products in the mixed feed stream into the first HDO reactor by breaking carbon-carbon and carbon oxygen bonds. The reactions are exothermic, but this may vaporize some of the products and water, thereby tempering the overall temperature rise. The reactions in the first HDO reactor also generally increase the thermal stability of the product of the first HDO reactor (and, correspondingly, the feed stream of the second HDO reactor) as compared to the feed stream into the first HDO reactor. Correspondingly, by separating the HDO reactions between a first and a second reactor, thermal stability can be improved for the inlet stream for the second HDO reactor (i.e., the product stream of the first HDO reactor), which may permit a corresponding increase in reaction temperatures in the second HDO reactor relative to the first HDO reactor.

The product from second HDO reactor can be separated for recycle of vapor, e.g., may be flashed in a high-pressure separator with the resulting vapor phase being subsequently condensed after some heat recovery into the first HDO reactor feed stream (e.g., as discussed above) or other streams, as applicable. The liquid product can be let down in pressure (e.g., to about 300 psig) to flash off more vapors. These vapors may be sufficiently converted by upstream reactions and can be sent on (e.g., directly) into an AC reactor train. The remaining liquid may include primarily less-converted components, typically with more than three carbon atoms remaining and more than three oxygen atoms remaining.

The liquid fraction from this post-processing of the effluent stream from the second HDO reactor has a reduced concentration of water (e.g., about 10 wt % to about 25 wt %) and a portion of the liquid fraction can be recycled into the feed for the first HDO reactor, in combination with the HYD product and hydrogen. Such a recycle can enhance the thermal stability of the HDO reactor feed and can also increase the reactor performance due to the lower water content, which reduces the loss of reaction heat by vaporization.

A portion of the liquids from the flashed HDO reactor product can be sent downstream to AC processing, rather than being recycled to the HDO reactor feed. For example, this portion can be provided to a selective vaporizer, which may use hot AC recycle gas to strip out volatile components, with the least volatile components being purged from the bottom of the vessel and not sent forward for AC reactions.

### EXAMPLES

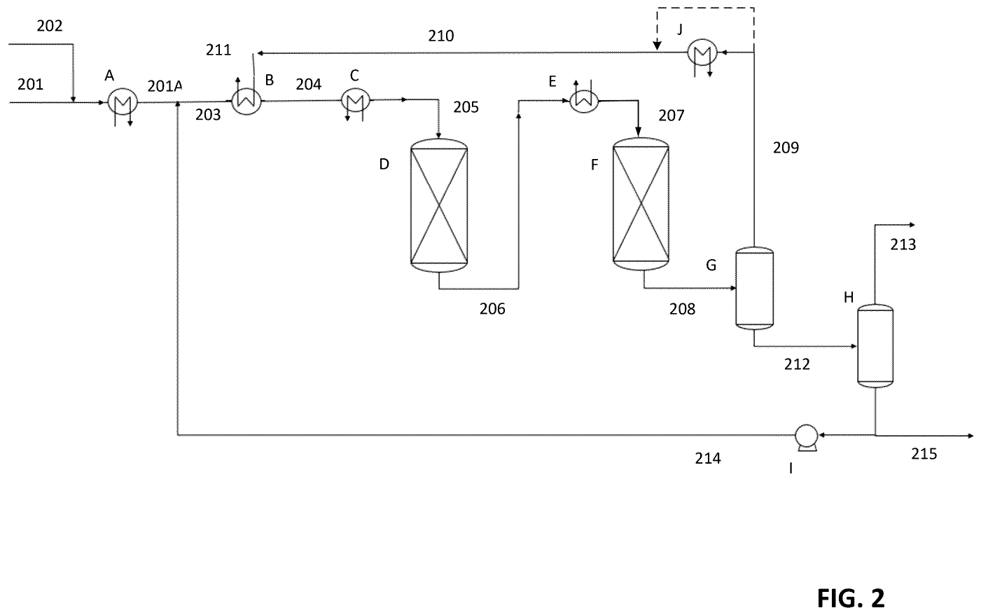

FIG. 2 illustrates an example configuration of an HDO reaction train according to an example implementation of the disclosed technology (e.g., as an example implementation of the HDO reactors 130, 140, with corresponding recycle stream 160). It should be recognized that although particular types of heat exchangers and separators are described in the example below, other suitable equipment may be similarly used as recognized by those of skill in the art.

In particular, a HYD product stream 201 is provided as an initial feed stream, which may be mixed with a hydrogen stream 202 (e.g., a recycled stream, a stream from APR, etc.). In some cases, the mixed feed stream 201-202 can be initially heated with a first HDO reactor feed preheater A (e.g., via non-mixing heat exchange with MP steam at 387° F. (197° C.) or other suitable temperature) to provide a heated feed stream 201A. In some implementations, the mixed and preheated stream 201A can then be further mixed with a liquid recycle stream 214, which may include a liquid fraction of output from the HDO reaction train and pressurized by a HDO reactor recycle pump I, as further discussed below. Generally, the liquid recycle stream 214 may include components with C₃₊ and O₃₊ composition, although a variety of species are possible.

The resulting mixed feed stream 203 can then be further heated at a second HDO reactor feed preheater B. In particular, the preheater B can provide heating of the feed stream 203 via non-mixing heat exchange with overhead vapor stream 210 as received from an HDO product separator G and cooled to a preheater temperature by an HDO product heat exchanger J before entering the preheater B. In some implementations, the resulting heated feed stream 204 can be further heated at a HDO reactor feed trim heater C (e.g., via non-mixing heat exchange with a low temperature hot oil at 550° F. (288° C.) or other suitable temperature). The cooled vapor stream 211 from the preheater B can then be passed to a further cooling train (not shown) or otherwise suitably processed.

The resulting fully preheated feed stream 205 can then pass through a first HDO reactor D, in which exothermic HDO reactions take place, resulting in a further heated first HDO effluent stream 206 (e.g., as an intermediate stream). The effluent stream 206 can then pass through an HDO reactor interbed heater E for further heating (e.g., via non-mixing heat exchange with high temperature hot oil with an inlet temperature of 625° F.).

Downstream of the heater E, the heated effluent stream 207 can then pass as an inlet stream to (and through) a second HDO reactor F, in which further exothermic HDO reactions take place, resulting in a second HDO effluent stream 208. The effluent stream 208 can then be passed to the HDO product separator G, which can result in an overhead vapor stream 209 and a liquid stream 212 (e.g., as an intermediate liquid stream).

As also noted above, the vapor stream 209 can be cooled at the HDO product heat exchanger J to provide the vapor stream 210 for preheating of the feed stream 203 at the preheater B. For example, the stream 209 can be cooled via non-mixing heat exchange with an HDO liquid product stream (e.g., at a temperature of about 393° F. (201° C.) to about 402° F. (206° C.)). The liquid stream 212 can be passed to an HDO product flash drum H, in which a let down in pressure causes a vapor stream 213 to flash off, leaving a liquid phase remainder. The vapor stream 213 can be provided to an AC reactor train as an AC feed stream (e.g., as the stream 145 in FIG. 1). The liquid phase remainder can be divided, as appropriate, between a further feed stream 215, which may be a feed stream for AC reactions (e.g., with further intermediate processing for vaporization, etc.), and the recycled liquid stream 214, which can be pressurized by an HDO product recycle pump I.

In an example process, the HDO reaction train illustrated in FIG. 2 can be operated so that the various streams have temperatures as listed below. In particular, it can be seen that the heated inlet stream 205 can be maintained at sufficiently low temperatures to ensure stability (i.e., to prevent thermal degradation), particularly in view of the mixing of the liquid recycle stream 214 with the mixture of the HYD product and hydrogen streams 201, 202 upstream of the first HDO reactor D and the heaters B, C.

| Stream | Temperature (start of run) | Temperature (end of run) |
|---|---|---|
| 201 | 123° C. (254° F.) | 172° C. (341° F.) |
| 201-202 (inlet | 113° C. (236° F.) | 149° C. (301° F.) |
| stream to | | |
| exchanger A) | | |
| 201A | 140° C. (284° F.) | 177° C. (350° F.) |
| 203 | 173° C. (344° F.) | 203° C. (399° F.) |
| 204 | 225° C. (437° F.) | 240° C. (464° F.) |
| 205 | 230° C. (446° F.) | 260° C. (500° F.) |
| 206 | 250° C. (482° F.) | 270° C. (518° F.) |
| 207 | 260° C. (500° F.) | 280° C. (536° F.) |
| 208 | 312° C. (594° F.) | 328° C. (622° F.) |
| 209 | 312° C. (594° F.) | 328° C. (622° F.) |
| 210 | 275° C. (527° F.) | 275° C. (527° F.) |
| 211 | 224° C. (436° F.) | 243° C. (470° F.) |
| 212 | 312° C. (594° F.) | 328° C. (622° F.) |
| 213 | 245° C. (473° F.) | 267° C. (512° F.) |
| 214/215 | 245° C. (473° F.) | 267° C. (512° F.) |

In some cases, to prevent undesired initial thermal degradation, it may be sufficient to ensure that the unmixed feed stream 201A (before introduction of the liquid HDO product recycle) does not exceed 464° F. (240° C.) and that the mixed feed stream 205 into the first HDO reactor D does not exceed a maximum inlet temperature of 500° F. (260° C.), although other configurations or streams may exhibit other maximum temperatures. In contrast, to prevent undesired intermediate thermal degradation, it may be sufficient to ensure that the feed stream 207 into the second HDO reactor F does not exceed a maximum inlet temperature of 554° F. (290° C.). In some cases, however, preheating may not necessarily approach the limits noted above. For example, the unmixed feed stream 201A in the example discussed above may be heated only to a maximum of 351° F. (177° C.) (e.g., using MP steam at 12 barg and 387° F. (197° C.)).

In testing, a thermal stability unit was arranged to test various streams in a BioForming process (i.e., a progression from HYD to HDO to AC) to determine upper temperature limits before fouling occurs. The unit consisted of a vertical heat exchanger followed by a filter (2μm) and then a dP cell measuring the pressure drop across the heat exchanger and the filter. Fluid was pumped upwards through the heat exchanger with a ½″ ID with a ⅛″ thermowell inside it. It was expected that at a high enough temperature, the material would start to break down and form larger solid foulant, which would cause a pressure drop to form across the heat exchanger or the filter. This increase in the pressure drop thus provides an indication of temperatures that are too high for that particular stream (i.e., that result in unwanted thermal degradation). Downstream of the filter, the fluid was pumped through a cooling water exchanger and through a back pressure regulator to hold the pressure in the heater at least above the boiling point of water for the pressure of operation.

In some relevant implementations, sorbitol is the main component in a HYD product stream. Accordingly, scoping runs were performed using a 60 wt % solution of sorbitol in water to determine suitable ranges for liquid velocity and residence time. A liquid feed rate of 2 ml/min was demonstrated to give a sensible time period for fouling to occur.

A protocol was developed which tested each material two times using temperature steps of 5-10° C. with holds of between 30 minutes and 4 hours. The results observed are presented below, with corresponding implications for maximum temperatures at which thermal degradation is not expected to occur (first temperature column), at which some thermal degradation is expected to initially occur (second temperature column), and at which substantial thermal degradation is expected to occur (third temperature column). In some implementations, it may thus be useful to control heat exchange operations (e.g., per the example discussed above) to prevent particular streams from exceeding any of the relevant temperatures, depending on the amount of thermal degradation that may be acceptable for a given process.

| | Max. Temp. | Temperature | Temperature |
|---|---|---|---|
| | Without | With Minor | With Major |
| | Fouling | Fouling First | Fouling |
| Stream | Observed | Observed | Observed |
| Description | (° C.) | (° C.) | (° C.) |
| HYD Inlet (2:1 | 180 | 190 | 200 |
| Recycle) | | | |
| HYD Inlet (1:1 | 188 | 194 | 201 |
| Recycle) | | | |
| HYD Product | 240 | — | 245 |
| HDO Reactor Inlet | 277 | — | 280 |
| Product Vaporiser | 350 | — | 350-400 |
| Bottoms (1800 psig) | | | |
| Product Vaporiser | 300 | — | — |
| Bottoms (250 psig) | | | |
| 95DE Corn Syrup | 134 | 140 | 190 |
| Selective Vaporiser | 250 | 300 | — |
| Bottoms | | | |

Thus, the disclosed systems and methods can provide for improved processing of biomass or other feedstocks. In particular, some examples, can include improved temperature control for inlet streams to one or more HDO reactors, with corresponding improvement in yield in the conversion of sugar feeds to liquid hydrocarbon (e.g., C₄₊) products and reduction coke deposits on downstream (e.g., AC) catalysts.

Unless otherwise specified or limited, the terms “about” and “approximately,” as used herein with respect to a reference value, refer to variations from the reference value of ±20% or less (e.g., ±15, ±10%, ±5%, etc.), inclusive of the endpoints of the range.

Unless otherwise specifically indicated, ordinal numbers are used herein for convenience of reference, based generally on the order in which particular components are presented in the relevant part of the disclosure. In this regard, for example, designations such as “first,” “second,” etc., generally indicate only the order in which a thus-labeled component is introduced for discussion and generally do not indicate or require a particular spatial, functional, temporal, or structural primacy or order. Relatedly, similar or identical components may be referred to with different ordinal numbers in different contexts.

1. A method for producing an oxygenate product, the method comprising:
(i) reacting a feed stream that includes an oxygenated hydrocarbon in a first hydrodeoxygenation (HDO) reactor with hydrogen in the presence of a first HDO catalyst to produce an intermediate stream, the feed stream having an inlet temperature at the first HDO reactor at which the oxygenated hydrocarbon is thermally stable;
(ii) reacting the intermediate stream in a second HDO reactor with hydrogen in the presence of a second HDO catalyst to produce an HDO product stream;
(iii) fractionating the HDO product stream to produce a first HDO vapor product stream and a first HDO liquid product stream;
(iv) heating the feed stream with the first HDO vapor product stream via a first heat exchanger upstream of the first HDO reactor; and
(v) recycling at least part of the first HDO liquid product stream to mix with the feed stream upstream of the first heat exchanger, to continue (i).

2. The method of claim 1, wherein the feed stream further comprises hydrogen.

3. The method of claim 2, wherein the feed stream including the hydrogen is preheated at a first preheater upstream of the first heat exchanger and upstream of mixing with the recycled at least part of the first HDO liquid product stream.

4. The method of claim 3, wherein the first preheater heats the feed stream to a temperature of less than about 240° C.

5. The method of claim 1, wherein the feed stream is further heated by a second preheater downstream of the first heat exchanger and upstream of the first HDO reactor.

6. The method of claim 1, wherein the inlet temperature of the feed stream at the first HDO reactor is about 200° C. to about 280° C.

7. The method of claim 6, wherein the inlet temperature of the feed stream at the first HDO reactor is less than about 277° C.

8. The method of claim 1, further comprising:
fractionating the first HDO liquid product stream to produce a second HDO vapor product stream and a second HDO liquid product stream, wherein one or more of the second HDO vapor product stream or the second HDO liquid product stream include the oxygenate product.

9. The method of claim 8, wherein recycling the at least part of the first HDO liquid product stream at (v) includes recycling at least part of the second HDO liquid product stream.

10. The method of claim 1, further comprising:
prior to (iv), cooling the first HDO vapor product stream, at a second heat exchanger, with a liquid product stream from one or more of the first or second HDO reactors.

11. The method of claim 10, further comprising:
selectively bypassing the second heat exchanger with a first part of the first HDO vapor product stream and remixing the first part of the first HDO vapor product stream with a second part of the first HDO vapor product stream that is cooled at the second heat exchanger.

12. A method for producing a C₄₊ compound, the method comprising:
producing a HDO vapor product stream and a HDO liquid product stream by:
(i) reacting a feed stream that includes an oxygenated hydrocarbon in a hydrodeoxygenation (HDO) reactor train with hydrogen in the presence of one or more HDO catalysts to produce an HDO product stream;
(ii) fractionating the HDO product stream to produce the HDO vapor product stream and the HDO liquid product stream; and
one or more of:
(iii) heating the feed stream with the first HDO vapor product stream via a first heat exchanger upstream of the HDO reactor train; or
(iv) recycling at least part of the first HDO liquid product stream to mix with the feed stream, to continue (i); and
reacting at least a portion of one or more of the HDO vapor product stream or the HDO liquid product stream in the presence of a condensation catalyst to produce the C₄₊ compound.

13. The method of claim 12, wherein producing the HDO product stream includes:
reacting the feed stream in a first HDO reactor with hydrogen in the presence of a first HDO catalyst to produce an intermediate stream; and
reacting the intermediate stream in a second HDO reactor with hydrogen in the presence of a second HDO catalyst to produce the HDO product stream.

14. The method of claim 13, further comprising heating the intermediate stream with a second heat exchanger upstream of the second HDO reactor.

15. The method of claim 12, wherein the method includes heating the feed stream with the first HDO vapor product stream via a first heat exchanger upstream of the HDO reactor train and recycling at least part of the first HDO liquid product stream to mix with the feed stream, to continue (i).

16. The method of claim 12, further comprising:
flashing the HIDO liquid product stream to produce a second HDO vapor product stream and a second HDO liquid product stream;
wherein the at least a portion of one or more of the HIDO vapor product stream or the HDO liquid product stream includes one or more of the second HDO vapor product stream or the second HDO liquid product stream.

17. The method of claim 16, wherein recycling at least part of the first HDO liquid product stream to mix with the feed stream includes recycling the second HDO liquid product stream to mix with the feed stream.

18. A system for producing a C₄₊ compound, the system comprising:
a first hydrodeoxygenation (HDO) reactor that includes a first HDO catalyst and is configured to receive a first HDO feed stream and to provide a first HDO effluent stream;
a second HDO reactor that includes a second HDO catalyst and is configured to receive the first HDO effluent stream from the first HDO reactor as a second HDO inlet stream and to provide a second HDO effluent stream; and
one or more of:
a recycle path configured to direct a liquid stream separated from the second HDO effluent stream into an initial feed stream to provide the first HDO feed stream; or
a first heat exchanger configured to heat the first HDO feed stream upstream of the first HDO reactor using a vapor stream separated from the second HDO effluent stream.

19. The system of claim 18, further comprising:
an HDO product separator configured to receive the second HDO effluent stream to separate the vapor stream from the second HDO effluent stream and provide an intermediate liquid stream from the second HDO effluent stream; and
an HDO product flash drum configured to receive the intermediate liquid stream to separate the liquid stream for the recycle path from a vapor product stream.

20. The system of claim 19, further comprising:
a hydrogenation (HYD) reactor train configured to provide a hydrogenation product as at least part of the first HDO feed stream; and
an acid condensation (AC) reactor train configured to receive the vapor product stream from the HDO product flash drum for condensation reactions to produce the C₄₊ compound.
